(19) United States
(12) Patent Application Publication
(10) Pub. No.: US 2020/0071255 A1
(43) Pub. Date: Mar. 5, 2020

(54) METHOD OF PRODUCING A DIARYL CARBONATE

(71) Applicant: SABIC GLOBAL TECHNOLOGIES B.V., Bergen op Zoom (NL)

(72) Inventor: Samir Anapat, Karnataka, Bangalore (IN)

(21) Appl. No.: 16/490,283

(22) PCT Filed: Mar. 9, 2018
(86) PCT No.: PCT/IB2018/051576

§ 371 Date: Aug. 30, 2019

(30) Foreign Application Priority Data
Mar. 9, 2017 (EP) 17159984.8

(51) Int. Cl. C07C 68/06

(52) U.S. Cl. CPC C07C 68/065

(57) ABSTRACT

A method of producing a diaryl carbonate, comprising: performing both a transesterification reaction and a disproportionation reaction within a reactive distillation column by introducing a feed stream comprising a dialkyl carbonate to the reactive distillation column at a point above a reboiler, and introducing an aromatic hydroxyl compound to the reactive distillation column; producing a diaryl carbonate, within the reactive distillation column; and withdrawing a bottom product stream comprising the diaryl carbonate from the reactive distillation column, preferably the diaryl carbonate has a purity of greater than or equal to 99.97 wt %.

BACKGROUND

Diphenyl carbonate (DPC) is a commercially valuable diaryl carbonate and acyclic carbonate ester. For example, diphenyl carbonate can be used as a monomer in combination with bisphenol A in the production of thermoplastic polycarbonate polymers. Such thermoplastic polycarbonates are durable engineering materials that can be worked, molded, and thermoformed with ease. Due to these favorable properties, polycarbonates find many useful applications, for example, in electronics. They are good electrical insulators and have heat-resistant and flame-retardant properties. As a result, polycarbonates can be used in various products associated with electrical and telecommunications hardware. They can also serve as dielectrics in high-stability capacitors. Furthermore, polycarbonates can be highly transparent to visible light, often with better light transmission than glass.

Various industrial processes can be used to obtain a diaryl carbonate, such as diphenyl carbonate, from a dialkyl carbonate (dimethyl carbonate, DMC) and an aromatic hydroxyl compound (phenol, PhOH). For example, a two-step process can be used to produce diphenyl carbonate. In the first step, dimethyl carbonate and phenol are reacted in the presence of a homogeneous or heterogeneous transesterification catalyst to obtain methyl phenyl carbonate (PMC) and methanol (MeOH) as shown in Formula (I). However, this reversible reaction can have an unfavorable equilibrium constant of approximately 0.002 at 200° C.

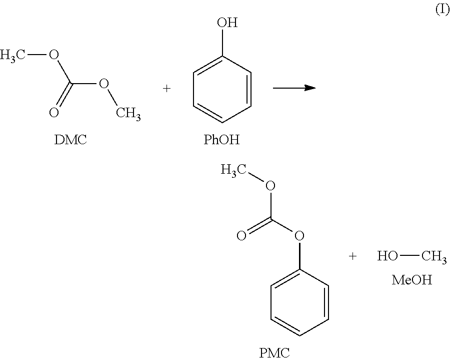

In the second step, as shown in Formula (II), methyl phenyl carbonate undergoes disproportionation to yield diphenyl carbonate and dimethyl carbonate. This reaction is also reversible with an unfavorable equilibrium constant of approximately 0.2 at 200° C.

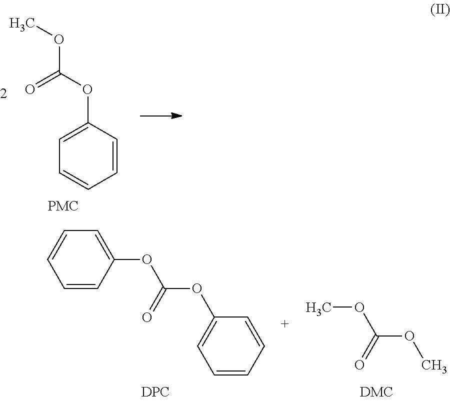

A significant amount of unwanted alkyl aryl ether impurity (for example, anisole) is also formed during the process as shown in Formula (III) and Formula (IV).

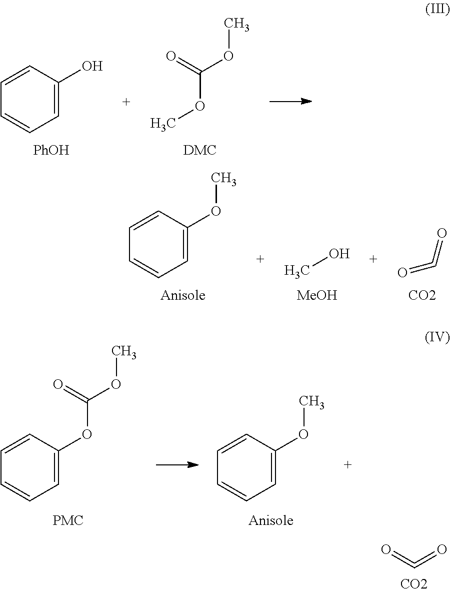

In industrial production methods, this chemistry is performed in at least two separate reactive distillation columns; the first one for transesterification and the second for disproportionation. This can then be followed by subsequent downstream unit operations for the recovery and recycle of unreacted dimethyl carbonate, phenol, methyl phenyl carbonate and catalyst. However, the use of multiple reactive distillation columns is highly inefficient, burdensome and costly.

Thus, there is a need for an efficient, high yield method of producing a diaryl carbonate that reduces the amount of impurities formed and reduces the amount of equipment required for production.

### SUMMARY

Disclosed herein is a method for producing diaryl carbonate.

A method of producing a diaryl carbonate, comprising: performing both a transesterification reaction and a disproportionation reaction within a reactive distillation column by introducing a feed stream comprising a dialkyl carbonate to the reactive distillation column at a point above a reboiler, and introducing an aromatic hydroxyl compound to the reactive distillation column; producing a diaryl carbonate, within the reactive distillation column; and withdrawing a bottom product stream comprising the diaryl carbonate from the reactive distillation column, preferably the diaryl carbonate has a purity of greater than or equal to 99.97 wt %.

The above described and other features are exemplified by the following drawings, detailed description, examples, and claims.

### BRIEF DESCRIPTION OF THE DRAWINGS

The following is a brief description of the drawings wherein like elements are numbered alike and which are presented for the purposes of illustrating the exemplary embodiments disclosed herein and not for the purposes of limiting the same.

FIG. 1 is a schematic diagram representing a method of producing a diaryl carbonate.

FIG. 2 (comparative) is a chart depicting the chemical equilibrium of a disproportionation reaction.

FIG. 3 is a chart depicting liquid mole fractions within the stages of a reactive distillation column.

FIG. 4 is a chart depicting the effects of DMC feed location on steam usage for a reactive distillation column.

### DETAILED DESCRIPTION

The method disclosed herein can provide an efficient, high yield method of producing a diaryl carbonate that reduces the amount of impurities formed and reduces the amount of equipment required for production and reduces power consumption. The process comprises performing both transesterification and disproportionation reactive distillation steps in a single column, by introducing the dialkyl carbonate at a stage above the reboiler (as opposed to directly into the reboiler) and preferably at or below the aromatic alcohol introduction point. This can create a zone of low dimethyl carbonate concentration between the feed stage and the reboiler within the reactive distillation column (e.g., less than or equal to 12 mole percent (mol %) at the feed stage and less than or equal to 1 mol % at the reboiler), which shifts the disproportionation reaction forward. The disproportionation reaction achieves significant conversion in the reboiler. This approach simplifies the overall process, reduces energy consumption, and reduces the total capital investment by eliminating an entire distillation column, reboiler and condenser. For example, surprisingly, steam usage can be reduced by greater than or equal to 15%, for example, greater than or equal to 19%. Furthermore, the formation of unwanted heavy impurities (i.e., materials that are less volatile than diphenyl carbonate), can be significantly decreased (e.g., decreased by greater than or equal to 95%); for example, where a conventional process would generate about 10 kilograms per hour (kg/h) heavies per 14,000 kg/h DPC, wherein the present process would generate about 0.5 kg/h heavies per 14,000 kg/h DPC.

The method for producing a diaryl carbonate can include passing one or more feed streams through a single reactive distillation column. For example, the feed stream(s) can comprise a dialkyl carbonate, for example, dimethyl carbonate, and an aromatic hydroxyl compound, for example, phenol. The feed stream(s) can further comprise a catalyst, for example, a transesterification catalyst. For example, a first feed stream comprising phenol and catalyst can be passed through a reactive distillation column. In addition to the first feed stream, a second feed stream comprising dimethyl carbonate can also be passed through the reactive distillation column simultaneously with the first feed stream.

The catalyst can be homogeneous or heterogeneous. For example, the catalyst can be any catalyst that achieves greater than or equal to 90% of an equilibrium transesterification and/or disproportionation conversion in less than or equal to 120 minutes in a batch reactor. Examples of homogeneous and heterogeneous catalysts include oxides (e.g., MoOₓ, CrOₓ, WOₓ, VOₓ, TiO₂, ZrO₂, CdO, Sm₂O₃, Fe₂O₃, Ga₂O, CuO) on supports (e.g., Al₂O₃, SiO₂, MgO, C, ZSM-5) as described by W. B. Kim and J. S. Lee in Catalysis Letters (1999), Volume 59, Page 83; and by Z. Fu and Y. Ono in the Journal of Molecular Catalysis A (1997), Volume 118, Page 293. For example, MoO₃/SiO₂ and/or TiO₂/SiO₂, which show selectivity towards PMC and DPC, can be used. A combination of both homogeneous and heterogeneous catalysts materials can also be used, for example, n-Bu₂SnO in combination with copper compounds (e.g., CuO, Cu₂O, CuCl, CuBr, Cul) as described by Zhi Ping Du, et al. in Advanced Materials Research (2012), Volumes 396-398, Pages 759-763.

Further examples of transesterification catalysts include alkali metals and alkaline earth metals such as lithium, sodium, potassium, magnesium, calcium, and barium; basic compounds of alkali metals and alkaline earth metals such as hydrides, hydroxides, alkoxides, aryloxides, and amides; basic compounds of alkali metals and alkaline earth metals such as carbonates, bicarbonates, and organic acid salts; tertiary amines such as triethylamine, tributylamine, trihexylamine, and benzyldiethylamine; nitrogen-containing heteroaromatic compounds such as N-alkylpyrroles, N-alkylindoles, oxazoles, N-alkylimidazoles, N-alkylpyrazoles, oxadiazoles, pyridines, quinolines, isoquinolines, acridines, phenanthrolines, pyrimidines, pyrazine, and triazines; cyclic amidines such as diazobicycloundecene (DBU) and diazobicyclononene (DBN); tin compounds such as tributylmethoxytin, dibutyldiethoxytin, dibutylphenoxytin, diphenylmethoxytin, dibutyltin acetate, tributyltin chloride, and tin 2-ethylhexanoate; zinc compounds such as dimethoxyzinc, diethoxyzinc, ethylenedioxyzinc, and dibutoxyzinc; aluminum compounds such as aluminum trimethoxide, aluminum triisopropoxide, and aluminum tributoxide; titanium compounds such as tetramethoxytitanium, tetraethoxytitanium, tetrabutoxytitanium, dichlorodimethoxytitanium, tetraisopropoxytitanium, titanium acetate, and titanium acetylacetonate; phosphorus compounds such as trimethylphosphine, triethylphosphine, tributylphosphine, triphenylphosphine, tributylmethylphosphonium halides, trioctylbutylphosphonium halides, and triphenylmethylphosphonium halides; zirconium compounds such as zirconium halides, zirconium acetylacetonate, zirconium alkoxides, and zirconium acetate; and lead and lead-containing compounds, for example, lead oxides such as PbO, PbO₂, and Pb₃O₄, lead sulfides such as PbS, Pb₂S_{3,} and PbS₂, and lead hydroxides such as Pb(OH)₂, Pb₃O₂(OH)₂, Pb₂[PbO₂(OH)₂], and Pb₂O(OH)₂. For example, the catalyst can include titanium compounds such as titanium tetraphenoxide, titanium isopropylate, titanium tetrachloride, organotin compounds, and compounds of copper, lead, zinc, iron, and zirconium, and combinations comprising at least one of the foregoing.

The feed stage to the single reactive distillation column can be 3 to 15 stages, preferably 5 to 15 stages, above a reboiler of the single reactive distillation column. For example, a feed stage to the single reactive distillation column can be 10 stages above a reboiler. For example, a first feed stream comprising phenol and catalyst can be fed to a reactive distillation column at a feed stage of 5 to 30, for example, stage 7 to 20, for example, stage 10. In other words, a feed stage to the single reactive distillation column is between the condenser (first stage; stage 1), and the reboiler (final stage; e.g., stage 50). The reactive distillation column can comprise a feed stage “NF” and a total number of stages “NT”, such that:

NT-3<NF≤0.7 NT;

preferably,

NT-5<NF≤0.8NT.

wherein “NF” is great than or equal to 70% of “NT” and less than or equal to “NT” minus three. This means, that for a 50 stage column, the feed stage is between stage 35 and stage 47, for example between stage 40 and stage 45; for a 100 stage column, the feed stage is between stage 70 and stage 97, for example between stage 80 and stage 90.

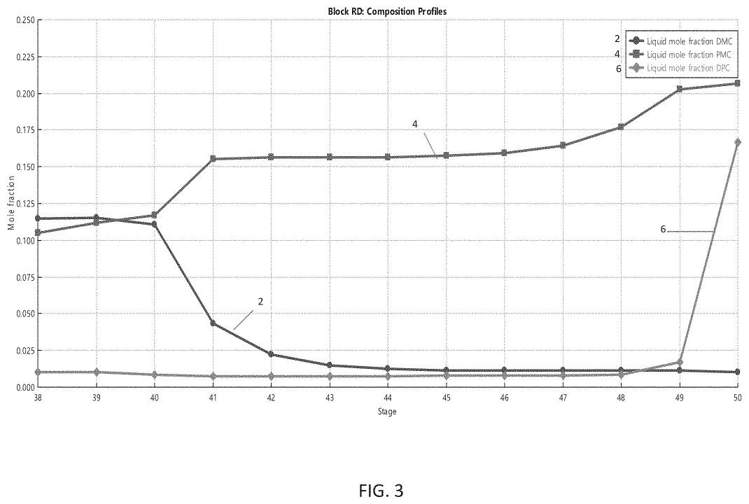

In addition to the first feed stream, a second feed stream comprising dimethyl carbonate can also be fed to a reactive distillation column at a feed stage of 50 to 70, for example, stage 60; or the second feed stream comprising dimethyl carbonate can be fed to a reactive distillation column at a feed stage of 35 to 48, for example, stage 40 (e.g., out of 50 stages). Accordingly, a zone of low dimethyl carbonate concentration can be formed between the feed stage for the second feed stream and the final stage (i.e., the reboiler) of the reactive distillation column. For example, a concentration of dimethyl carbonate can be less than or equal to 12 mol % at the second feed stage and gradually decrease to less than or equal to 1 mol % at the final stage. This trend can be seen in FIG. 3.

Both a transesterification reaction and a disproportionation reaction can occur within a single reactive distillation column. For example, a diaryl carbonate can be produced within the single reactive distillation column, for example, a diphenyl carbonate product. For example, greater than or equal to 50 kilomoles per hour of diphenyl carbonate can be produced, for example, greater than or equal to 65 kilomoles per hour. Less than or equal to 0.01 kilomoles per hour of heavy impurities can be produced within the single reactive distillation column, for example, less than or equal to 0.006 kilomoles per hour of heavy impurities. Less than or equal to 40 parts per million by weight (ppm) heavy impurities can be present in a diphenyl carbonate stream of the present method, as compared to greater than or equal to 700 ppm in a diphenyl carbonate stream of a conventional method. Less than or equal to 0.005 mol % of heavy impurities per mole of diphenyl carbonate can be produced within the single reactive distillation column, for example, less than or equal to 0.002 mol %. As used herein, “heavies” (also referred to as high boilers; include those compounds that have a boiling point higher than the boiling point of DPC under the conditions of the unit they are located in).

A temperature within the single reactive distillation column can be 150° C. to 250° C. For example, a temperature of a bottom portion (i.e., in the reboiler) of the single reactive distillation column can be less than or equal to 250° C., for example, less than or equal to 240° C. A pressure within the single reactive distillation column can be 150 kiloPascals (kPa) to 300 kiloPascals.

A concentration of catalyst in a bottom portion (i.e., in the reboiler) of the single reactive distillation column can be less than or equal to 5 weight percent (wt %), for example, less than or equal to 3 wt %. A concentration of diphenyl carbonate in a bottom portion (i.e., a reboiler) of the single reactive distillation column can be less than or equal to 25 wt %. Less than or equal to 4 kilograms of pressurized steam can be used per kilogram of diphenyl carbonate produced, for example, less than or equal to 3.9 kilograms of pressurized steam can be used per kilogram of diphenyl carbonate produced. The pressurized steam can be saturated steam at 3,500 kPa to 4,500 kPa, for example, 4,000 kPa, and at 200° C. to 300° C., for example, 250° C.

A top product stream comprising unreacted dimethyl carbonate and methanol can be produced and withdrawn from the reactive distillation column. The top product stream can be passed through a methanol distillation column. For example, the methanol distillation column can isolate and purge methanol from the top product stream of the reactive distillation column. For example, a methanol purge stream can be produced as a low-boiling azeotrope and withdrawn from a top portion of the methanol distillation column. A dimethyl carbonate recycle stream can also be produced and withdrawn from a bottom portion of the methanol distillation column. For example, the dimethyl carbonate recycle stream can be recycled back to the feed stream(s).

A side draw product stream comprising anisole can be produced and withdrawn from the reactive distillation column. The side draw product stream can comprise greater than or equal to 50% anisole, for example, greater than or equal to 75 mol %, or greater than or equal to 95 mol % anisole, for example, greater than or equal to 99 mol % anisole, or greater than or equal to 99.9 mol % anisole. The side draw product stream can also comprise less than or equal to 5 mol % phenol, for example, less than or equal to 1 mol % phenol, or less than or equal to 0.1 mol % phenol. For example, maintaining a low phenol concentration of 1 mol % in a top portion of the single reactive distillation column can result in a side draw product of 99 mol % anisole. The side draw product stream can be passed through an anisole distillation column. For example, the anisole distillation column can isolate and purge anisole from the side draw stream of the reactive distillation column. For example, an anisole purge stream can be produced and withdrawn from a bottom portion of the anisole distillation column. A top product stream can also be produced and withdrawn from the anisole distillation column. This top product stream can be recycled to the methanol distillation column.

A bottom product stream comprising diphenyl carbonate can be produced and withdrawn from the reactive distillation column. For example, the bottom product stream can be passed through a catalyst processing unit. For example, the catalyst processing unit can comprise a flash drum and/or a wiped film evaporator. The catalyst processing unit can isolate and purge catalyst and heavy impurities from the bottom product stream. For example, a stream comprising catalyst and heavy impurities can be produced and withdrawn from the catalyst processing unit. The catalyst can be further isolated and recycled back to the feed stream(s).

Accordingly, a stream purged of catalyst and heavy impurities can be produced and withdrawn from a top portion of the catalyst processing unit. This stream can be passed through a diphenyl carbonate distillation column. For example, the diphenyl carbonate distillation column can isolate and produce a purified diphenyl carbonate product. For example, a stream comprising purified diphenyl carbonate can be produced and withdrawn from a bottom portion of the diphenyl distillation column. For example, the purified diphenyl carbonate product stream can comprise greater than or equal to 99% diphenyl carbonate, for example, the purified diphenyl carbonate product stream can comprise greater than or equal to 99.99% diphenyl carbonate. A recycle stream comprising phenol can be produced and withdrawn as a top product from the diphenyl distillation column. For example, the phenol recycle stream can be recycled back to feed stream(s). A side draw stream comprising methyl phenyl carbonate can also be produced and withdrawn from the diphenyl carbonate distillation column. For example, the side draw stream can be recycled back to the reactive distillation column.

A more complete understanding of the components, processes, and apparatuses disclosed herein can be obtained by reference to the accompanying drawings. These figures (also referred to herein as “FIG.”) are merely schematic representations based on convenience and the ease of demonstrating the present disclosure, and are, therefore, not intended to indicate relative size and dimensions of the devices or components thereof and/or to define or limit the scope of the exemplary embodiments. Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

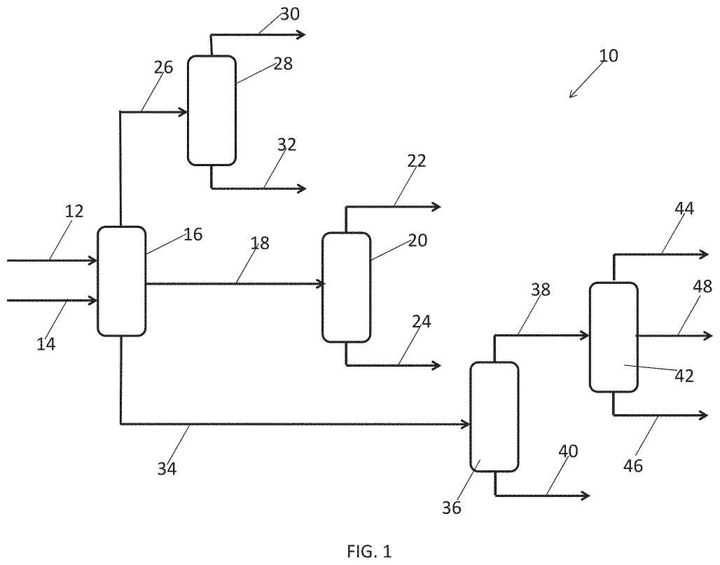

Referring now to FIG. 1, this simplified schematic diagram represents a method 10 for producing a diaryl carbonate. The method 10 can include passing a feed stream 12 comprising phenol and catalyst through a reactive distillation column 16. In addition to feed stream 12, a feed stream 14 comprising dimethyl carbonate can also be passed through the reactive distillation column 16. Both a transesterification reaction and a disproportionation reaction can occur within the reactive distillation column 16.

A top product stream 26 comprising unreacted dimethyl carbonate and methanol can be produced and withdrawn from the reactive distillation column 16. The top product stream 26 can be passed through methanol distillation column 28. The methanol distillation column 28 can isolate and purge methanol from the top product stream 26 of the reactive distillation column 16. For example, a methanol purge stream 30 can be produced and withdrawn from a top portion of the methanol distillation column. A dimethyl carbonate recycle stream 32 can also be produced and withdrawn from a bottom portion of the methanol distillation column 28. For example, the dimethyl carbonate recycle stream 32 can be recycled back to the feed stream 14.

A side draw product stream 18 comprising anisole can be produced and withdrawn from the reactive distillation column 16. The side draw product stream 18 can be passed through an anisole distillation column 20. The anisole distillation column 20 can isolate and purge anisole from the side draw stream 18 of the reactive distillation column 16. For example, an anisole purge stream 24 can be produced and withdrawn from a bottom portion of the anisole distillation column 20. A top product stream 22 can also be produced and withdrawn from the anisole distillation column 20. This top product stream 20 can be recycled to the methanol distillation column 28.

A bottom product stream 34 comprising diphenyl carbonate can be produced and withdrawn from the reactive distillation column 16. The bottom product stream 34 can be passed through a catalyst processing unit 36. For example, the catalyst processing unit 36 can comprise a flash drum and a wiped film evaporator. The catalyst processing unit 36 can isolate and purge catalyst and heavy impurities from the bottom product stream 34. For example, stream 40 comprising catalyst and heavy impurities can be produced and withdrawn from a bottom portion of the catalyst processing unit 36. The catalyst can be further isolated and recycled back to the feed stream 12. A stream 38 purged of catalyst and heavy impurities can be produced and withdrawn from a top portion of the catalyst processing unit 36.

The stream 38 can be passed through a diphenyl carbonate distillation column 42. The diphenyl carbonate distillation column 42 can isolate and produce a purified diphenyl carbonate product. For example, a stream 46 comprising purified diphenyl carbonate can be produced and withdraw from a bottom portion of the diphenyl distillation column 42. A recycle stream 44 comprising phenol can be produced and withdrawn as a top product from the diphenyl distillation column 42. The phenol recycle stream 44 can be recycled back to feed stream 12. A side draw stream 48 comprising methyl phenyl carbonate can also be produced and withdrawn from the diphenyl carbonate distillation column 42. The side draw stream 48 can be recycled back to the reactive distillation column 16.

The following examples are merely illustrative of the method of producing a diaryl carbonate disclosed herein and is not intended to limit the scope hereof.

### EXAMPLES

### Example 1

### Comparative

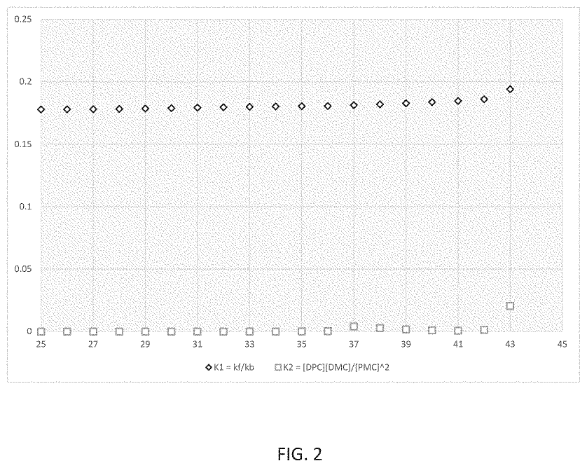

Simulations are conducted using a steady state process model in ASPEN PLUS (commercial process engineering software). Comparative Example 1 represents a DPC production process similar to that seen in U.S. Pat. No. 7,141,641, wherein a series of three distillation columns are used, including a disproportionation column (as opposed to the present method, wherein both transesterification and disproportionation are achieved within a single column). The tray-by-tray chemical equilibrium of a disproportionation column is calculated. FIG. 2 depicts the chemical equilibrium of the disproportionation reaction in the column. FIG. 2 shows that the column is very inefficient from a reaction perspective; there is minimal reaction occurring on the trays of the column. For example, chemical equilibrium should be achieved on each tray of the column. For example, the kinetic ratio represented by Formula (V):

$$\frac{[ DPC ] [ DMC ]}{{[ PMC ]}^{2}}$$

should be equal to the chemical reaction equilibrium constant represented by Formula (VI):

$${K}_{eq} = \frac{{k}_{f}}{{k}_{b}}$$

where brackets “[ ]” denote concentration in kilomoles per cubic meter (kmol/m³) and k_{f}, k_{b} are the forward and reverse reaction rate constants respectively. As FIG. 2 shows however, K_{eq} is approximately 0.2. This indicates that the compositions on each stage of the column are far removed from equilibrium. Instead, most of the reaction happens in the reboiler. This is because the trays have a very small reaction holdup and the reboiler has a significant reaction holdup.

### Example 2

Simulations are conducted using a steady state process model in ASPEN PLUS (commercial process engineering software) and in accordance with the method disclosed herein for producing diphenyl carbonate (DPC) as depicted in FIG. 1. The reactive distillation (RD) column (16) is modeled as an equilibrium RADFRAC block with reactions modeled as elementary power law kinetics. Instantaneous mass transfer and the attainment of vapor-liquid equilibrium (VLE) at each stage are assumed. The transesterification rate is represented by Formula (VII):

$${r}_{te} = [ Cat ] ( {k}_{f , te} [ PhOH ] [ DMC ] - \frac{{k}_{f , te}}{{K}_{eq , te}} [ PMC ] [ MeOH ] )$$

and the disproportionation rate is represented by Formula (IX):

$${r}_{dp} = [ Cat ] ( {k}_{f , dp} [ PMC ] [ PMC ] - \frac{{k}_{f , dp}}{{K}_{eq , dp}} [ DPC ] [ DMC ] )$$

VLE is modeled using the NRTL activity coefficient model with physical properties for all the pure components except PMC & DPC from the standard Aspen Properties databank (PURE35 and NIST). The RD column is modeled with 40-60 stages, with varying feed locations for phenol (2-20) and DMC (2-10 stages from the bottom). The location of the side-draw for anisole is varied between stages 2-5.

The simulation is formulated as a constrained optimization problem with constraints on the RD column. Phenol and a Ti(OPh)₄ catalyst are fed to the column at stage 10, DMC is fed at stage 40, and recycled PMC is fed at stage 41. A temperature at a top portion of the column is greater than or equal to 107° C. and a temperature at a bottom portion of the column is less than or equal to 240° C. Methanol and DMC are withdrawn from a top portion of the column. The anisole side draw is withdrawn at stage 2 and comprises less than or equal to 0.01 mol % phenol. DPC is withdrawn as bottom product at a rate of greater than or equal to 65 kilomoles per hour and comprises less than or equal to 2 wt % catalyst. The final stage of the reactor is stage 50.

The downstream catalyst processing unit (36) and the DPC distillation column (42), as depicted in FIG. 1, are modeled using RADFRAC blocks without reactions. The methanol distillation column (28) and anisole distillation column (20) are modeled as RADFRAC blocks without reactions. The phenol, PMC, DMC and catalyst recycle streams are connected and the entire flow sheet is optimized to minimize the specific steam usage (kg of steam (saturated at 4,000 kPa)/kg of DPC) while honoring the constraints around the RD column and additional constraints due to product (e.g., azeotrope, anisole and DPC) purity. Integer decision variables (i.e., stages, feed and product locations) are changed manually and the flow sheet is optimized for several combinations of the integer variables.

TABLE 1
Comparative Results

| | Unit | Example 1 | Example 2 |
|---|---|---|---|
| DPC produced | kg/hr | 15,505 | 15,524 |
| Phenol feed | kg/hr | 13,860 | 14,139 |
| DMC feed | kg/hr | 8,634 | 9,277 |
| Makeup catalyst | kg/hr | 34 | 31 |
| Total reactive holdup | m³ | 143.69 | 105 |
| Steam usage | ton/hr | 78.46 | 63.26 |
| Steam savings | % | — | 19.37 |

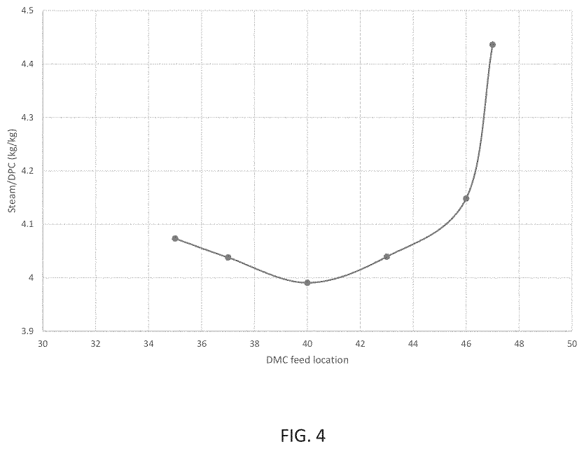

Table 1 compares the results of Comparative Example 1 (U.S. Pat. No. 7,141,641; three reactive columns) and Example 2 of the present method (single column). As shown, significant and unexpected improvements are achieved. For example, steam usage can be reduced by greater than or equal to 15%, for example, greater than or equal to 19%. FIG. 4 demonstrates the unexpected sensitivity of steam usage to the DMC feed stage (wherein stage 1 is the condenser and stage 50 is the reboiler) over the range of 5 to 15 stages above the reboiler. As shown, the steam usage increases rapidly as the DMC feed stage gets closer to the reboiler.

There are many benefits realized with the present process. By employing the single reactive distillation column to perform both the transesterification reaction and disproportionation reactions, at least one of the following advantages is achieved: i) the amount of DPC at the bottom of the column is reduced, e.g., by greater than or equal to 50%, and even by greater than or equal to 60% (for example, reduced from 65 wt % to about 25 wt %); ii) a reduction in the amount of impurities (also referred to as heavy impurities), e.g., by orders of magnitude (e.g., greater than or equal to 87%, and even by greater than or equal to 95%), for example, from 0.442 kilomoles per hour (kmol/hr) to 0.006 kmol/hr; iii) reduced high pressure steam (e.g., saturated steam at 4,000 kPa) usage (e.g., from 4.7 kilogram steam per kilogram diaryl carbonate to 3.9 kilogram steam per kilogram diaryl carbonate); and iv) elimination of a column, reboiler, and condenser from the process.

Set forth below are some embodiments of the present method.

Aspect 1: A method of producing a diaryl carbonate, comprising: performing both a transesterification reaction and a disproportionation reaction within a single reactive distillation column by introducing a feed stream comprising a dialkyl carbonate (preferably dimethyl carbonate) to the reactive distillation column at a point above a reboiler, preferably greater than or equal to 3 stages, preferably greater than or equal to 5 stages, more preferably greater than or equal to 8 stages, above the reboiler, and introducing an aromatic hydroxyl compound (preferably phenol) to the reactive distillation column; producing a diaryl carbonate, preferably diphenyl carbonate, within the reactive distillation column; and withdrawing a bottom product stream comprising the diaryl carbonate, preferably diphenyl carbonate, from the reactive fdistillation column, preferably the diaryl carbonate has a purity of greater than or equal to 99.97 wt %, or greater than or equal to 99.99 wt %.

Aspect 2: A method of producing a diaryl carbonate, comprising: performing both a transesterification reaction and a disproportionation reaction within a single reactive distillation column by introducing an aromatic hydroxyl compound (preferably phenol) to the reactive distillation column, and introducing a feed stream comprising a dialkyl carbonate (preferably dimethyl carbonate) to the reactive distillation column at a feed stage “NF”, wherein the feed stage “NF” satisfies the formula

NT-3≤NF≤0.7 NT, preferably NT-5≤NF≤0.8 NT.

wherein “NT” is the total number of stages; producing a diaryl carbonate, preferably diphenyl carbonate, within the reactive distillation column; and withdrawing a bottom product stream comprising the diaryl carbonate, preferably diphenyl carbonate, from the reactive distillation column.

Aspect 3: The method of Aspect 1 or Aspect 2, wherein the feed stream further comprises a catalyst, wherein the catalyst comprises alkali metals, tertiary amines, heteroaromatic nitrogen compounds, cyclic amidines, tin compounds, zinc compounds, aluminum compounds, titanium compounds, phosphorus compounds, zirconium compounds, lead compounds, or a combination comprising at least one of the foregoing.

Aspect 4: The method of any of the preceding aspects, wherein the diaryl carbonate comprises diphenyl carbonate.

Aspect 5: The method of any of the preceding aspects, wherein a feed stage to the reactive distillation column is 5 to 15 stages above a reboiler of the reactive distillation column; preferably, wherein a feed stage to the reactive distillation column is 10 stages above a reboiler of the reactive distillation column.

Aspect 6: The method of any of the preceding aspects, wherein a temperature within the reactive distillation column is 150° C. to 250° C. and a pressure within the reactive distillation column is 150 kPa to 300 kPa.

Aspect 7: The method of any of the preceding aspects, further comprising withdrawing a top stream comprising dimethyl carbonate and methanol from the reactive distillation column.

Aspect 8: The method of any of the preceding aspects, further comprising withdrawing a side draw product stream comprising anisole from the reactive distillation column.

Aspect 9: The method of Aspect 8, wherein the side draw product stream comprises greater than or equal to 50% anisole, preferably greater than or equal to 95% anisole.

Aspect 10: The method of any of the preceding aspects, further comprising passing the bottom product stream of the reactive distillation column through a catalyst processing unit.

Aspect 11: The method of Aspect 10, wherein the catalyst processing unit comprises a flash drum and/or a wiped film evaporator.

Aspect 12: The method of any of any of the preceding aspects, further comprising passing the bottom product stream of the reactive distillation column through a diphenyl carbonate distillation column.

Aspect 13: The method of any of the preceding aspects, wherein a concentration of phenol in a top portion of the reactive distillation column is less than or equal to 1 mol %; preferably less than or equal to 0.01 mol %.

Aspect 14: The method of any of the preceding aspects, wherein a concentration of catalyst in a bottom portion of the reactive distillation column is less than or equal to 5 wt %; preferably, wherein a concentration of catalyst in a bottom portion of the reactive distillation column is less than or equal to 3 wt %.

Aspect 15: The method of any of the preceding aspects, wherein a temperature within a bottom portion of the reactive distillation column is less than or equal to 250° C.

Aspect 16: The method of any of the preceding aspects, wherein less than or equal to 0.005 mol % of heavy impurities per mole of diphenyl carbonate are produced within the reactive distillation column; preferably, less than or equal to 0.002 mol %.

Aspect 17: The method of any of the preceding aspects, wherein less than or equal to 4 kg of pressurized steam (e.g. saturated at 4,000 kPa) is used per kilogram of diaryl carbonate produced; preferably, wherein less than or equal to 3.9 kilograms of pressurized steam is used per kilogram of diaryl carbonate produced.

Aspect 18: The method of any of the preceding aspects, wherein a temperature of a bottom portion of the reactive distillation column is less than or equal to 250° C.; preferably, wherein a temperature of a bottom portion of the reactive distillation column is less than or equal to 240° C.

Aspect 19: The method of any of the preceding aspects, further comprising: withdrawing a top product stream comprising dimethyl carbonate and methanol from the reactive distillation column; withdrawing a side draw product stream comprising anisole from the reactive distillation column, wherein the side draw product stream comprises greater than or equal to 50% anisole, preferably greater than or equal to 95% anisole; withdrawing a bottom product stream comprising the diphenyl carbonate product from the reactive distillation column; passing the bottom product stream of the reactive distillation column through a catalyst processing unit; and passing the bottom product stream of the reactive distillation column through a diphenyl carbonate distillation column.

Aspect 20: The method of any of the preceding aspects, wherein the reactive distillation column has a dialkyl carbonate concentration at the feed stage of less than or equal to 15 mol %, preferably less than or equal to 12 mol %.

Aspect 21: The method of any of the preceding Aspects, wherein the reactive distillation column has a dialkyl carbonate concentration at the reboiler of less than or equal to 4 mol %, preferably less than or equal to 2 mol %, more preferably less than or equal to 1 mol %, or less than or equal to 0.5 mol %.

Aspect 22: The method of any of the preceding aspects, wherein the reactive distillation column comprises a feed stage “NF” and a total number of stages “NT”, wherein

NT-3≤NF≤0.7NT, preferably NT-5≤NF≤0.8 NT.

Aspect 23: The method of any of the preceding aspects, wherein the dialkyl carbonate is dimethyl carbonate and/or the aromatic hydroxyl compound is phenol.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. “Combination” is inclusive of blends, mixtures, alloys, reaction products, and the like. Furthermore, the terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context. “Or” means “and/or.” The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and to “one aspect”, “another aspect”, “an aspect, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The modifier “about” used in connection with a quantity is inclusive of the stated value and has the meaning dictated by the context (e.g., includes the degree of error associated with measurement of the particular quantity). The notation “±10%” means that the indicated measurement can be from an amount that is minus 10% to an amount that is plus 10% of the stated value. The terms “front”, “back”, “bottom”, and/or “top” are used herein, unless otherwise noted, merely for convenience of description, and are not limited to any one position or spatial orientation. “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this invention belongs. A “combination” is inclusive of blends, mixtures, alloys, reaction products, and the like.

All cited patents, patent applications, and other references are incorporated herein by reference in their entirety. However, if a term in the present application contradicts or conflicts with a term in the incorporated reference, the term from the present application takes precedence over the conflicting term from the incorporated reference

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

What is claimed is:

1. A method of producing a diaryl carbonate, comprising:
performing both a transesterification reaction and a disproportionation reaction within a single reactive distillation column by introducing a feed stream comprising a dialkyl carbonate and introducing an aromatic hydroxyl compound to the reactive distillation column;
producing a diaryl carbonate within the reactive distillation column; and
withdrawing a bottom product stream comprising the diaryl carbonate from the reactive distillation column.

2. The method of claim 1, wherein the dialkyl carbonate comprises dimethyl carbonate, the aromatic hydroxyl compound comprises phenol, the diaryl carbonate comprises diphenyl carbonate, or combinations thereof.

3. The method of claim 1, wherein a feed stage to the reactive distillation column is greater than or equal to 5 above a reboiler of the reactive distillation column.

4. The method of claim 1, wherein a temperature within the reactive distillation column is 150° C. to 250° C. and a pressure within the reactive distillation column is 150 kPa to 300 kPa.

5. The method of claim 1, further comprising withdrawing a top stream comprising dimethyl carbonate and methanol from the reactive distillation column.

6. The method of claim 1, further comprising withdrawing a side draw product stream comprising anisole from the reactive distillation column, and wherein the side draw product stream comprises greater than or equal to 50% anisole.

7. The method of claim 1, further comprising passing the bottom product stream of the reactive distillation column through a catalyst processing unit, and wherein the catalyst processing unit comprises a flash drum and/or a wiped film evaporator.

8. The method of claim 1, further comprising passing the bottom product stream of the reactive distillation column through a diphenyl carbonate distillation column.

9. The method of claim 1, wherein a concentration of phenol in a top portion of the reactive distillation column is less than or equal to 1 mol %.

10. The method of claim 1, wherein a concentration of catalyst in a bottom portion of the reactive distillation column is less than or equal to 5 wt %.

11. The method of claim 1, wherein a temperature within a bottom portion of the reactive distillation column is less than or equal to 250° C.

12. The method of claim 1, wherein less than or equal to 0.005 mol % of heavy impurities per mole of diphenyl carbonate are produced within the reactive distillation column.

13. The method of claim 1, wherein less than or equal to 4 kg of pressurized steam is used per kilogram of diaryl carbonate produced.

14. The method of claim 1, wherein the reactive distillation column has a dialkyl carbonate concentration at the feed stage of less than or equal to 15 mol % and/or a dialkyl carbonate concentration at the reboiler of less than or equal to 2 mol %.

15. The method of claim 1, further comprising
withdrawing a top product stream comprising dimethyl carbonate and methanol from the reactive distillation column;
withdrawing a side draw product stream comprising anisole from the reactive distillation column, wherein the side draw product stream comprises greater than or equal to 95% anisole;
withdrawing a bottom product stream comprising the diphenyl carbonate product from the reactive distillation column;
passing the bottom product stream of the reactive distillation column through a catalyst processing unit; and
passing the bottom product stream of the reactive distillation column through a diphenyl carbonate distillation column.

16. A method of producing a diaryl carbonate, comprising:
performing both a transesterification reaction and a disproportionation reaction within a single reactive distillation column by introducing an aromatic hydroxyl compound to the reactive distillation column, and introducing a feed stream comprising a dialkyl carbonate to the reactive distillation column at a feed stage “NF”, wherein the feed stage “NF” satisfies the formula
NT-3≤NF≤0.7 NT, preferably NT-5≤NF≤0.8 NT.
wherein “NT” is the total number of stages;
producing a diaryl carbonate within the reactive distillation column; and
withdrawing a bottom product stream comprising the diaryl carbonate from the reactive distillation column.

17. The method of claim 1, wherein the diaryl carbonate has a purity of greater than or equal to 99.97 wt %.

18. The method of claim 6, wherein the side draw product stream comprises greater than or equal to 95% anisole.

19. The method of claim 9, wherein a concentration of phenol in a top portion of the reactive distillation column is less than or equal to 0.01 mol %.

20. The method of claim 16, wherein the dialkyl carbonate comprises dimethyl carbonate, the aromatic hydroxyl compound comprises phenol, the diaryl carbonate comprises diphenyl carbonate, or combinations thereof.
